(19) United States
(12) Patent Application Publication
(10) Pub. No.: US 2025/0104246 A1
(43) Pub. Date: Mar. 27, 2025

(54) METHODS, DEVICES AND SYSTEMS FOR PREPARING MEDICAL IMAGE DATA OF THE HEART FOR A DEFORMATION ANALYSIS

(71) Applicant: Siemens Healthineers AG, Forchheim (DE)

(72) Inventors: Klaus FISCHER, Erlangen (DE); Michael WELS, Bamberg (DE); Felix DENZINGER, Herzogenaurach (DE)

(73) Assignee: Siemens Healthineers AG, Forchheim (DE)

(21) Appl. No.: 18/892,740

(22) Filed: Sep. 23, 2024

(30) Foreign Application Priority Data
Sep. 26, 2023 (DE) 10 2023 209 370.7

(51) Int. Cl. G06T 7/11; A61B 6/00; A61B 6/50; G06T 7/00; G06T 7/174; G06T 7/20

(52) U.S. Cl. CPC G06T 7/11; A61B 6/481; A61B 6/503; A61B 6/54; G06T 7/0012; G06T 7/174; G06T 7/20; G06T 2207/10081; G06T 2207/30048; G06T 2207/30241

(57) ABSTRACT

A method is described for generating segmented, masked 4D image data of the heart. Furthermore, a cardiac deformation analysis method is described. An image data generating device is also described. Moreover, a medical imaging system is described.

### CROSS-REFERENCE TO RELATED APPLICATION(S)

The present application claims priority under 35 U.S.C. § 119 to German Patent Application No. 10 2023 209 370.7, filed Sep. 26, 2023, the entire contents of which is incorporated herein by reference.

### FIELD

One or more example embodiments relates to a method for generating segmented, masked 4D image data of the heart. Additionally, one or more example embodiments relates to a cardiac deformation analysis method. One or more example embodiments also relates to an image data generating device. Furthermore, the invention relates to a computed tomography system.

### RELATED ART

Image data is generated using modern imaging methods, which are used to visualize an imaged examination object. Image data or image data sets obtained in this way can also be used for other applications or analyses. This invention relates to another application or analysis of image data or image data sets obtained in this way.

Ischemic heart disease is one of the most common causes of death in the world. Blood flow is reduced by plaque deposits that narrow the circulatory vessels, restricting the supply to the heart muscle and limiting functionality. One way to identify the disease is by the volume of blood pumped by a heart contraction. This evaluation measures the heart's ejection fraction and is part of cardiac functional analysis (CFA). The disadvantage of this method is that it can only be used to identify the disease in later stages, as it is based on an indirect manifestation.

By contrast, cardiac strain analysis (CSA) facilitates identification of smaller, regionally restricted functional heart disorders even in seemingly asymptomatic patients. With cardiac deformation analysis, it is the cardiac motion that leads to a thickening and thinning of the myocardium, and thus to strain, that is measured. Accordingly, cardiac deformation analysis is a suitable supplementary method for identifying and localizing myocardial dysfunctions.

Non-invasive imaging modalities are usually used to capture cardiac deformations during the whole cardiac cycle. In the clinical environment, cardiac deformation analysis is mainly performed using echocardiography. It is known, however, that cardiac deformation analysis based on echocardiography is sensitive to the image recording quality. It is thus prone to error, and the results are not always reliable.

Another imaging method that is currently becoming increasingly popular in the field of cardiac deformation analysis is cardiac magnetic resonance tomography (CMRT). It offers improved image quality in terms of noise suppression and tissue differentiation. This facilitates precise CMRT-based CSA. However, CMRT is not widespread and does involve a time-consuming recording process. CMRT is described in M. M. Lamacie et al., “Quantifizierung der Myokardverformung durch verformbare, registrierungsbasierte Analyse von Cine-MRT: Validierung mit getaggter CMR,” European Radiology, vol. 29, no. 7, p. 3658-3668, 2019, doi: 10.1007/s00330-019-06019-9.

In cardiac imaging, computed tomography (CT) is primarily used for coronary angiography-a method for imaging coronary vessels using a contrast agent. Performing cardiac deformation analysis using cardiac CT imaging is an area of active research. Earlier approaches to cardiac deformation analysis using CT scans were moderately successful. They usually failed because motion estimation algorithms were not able to cope with low tissue contrasts in CT images. This is why CT cardiac deformation analysis is not yet performed in clinical practice.

Existing approaches for performing cardiac deformation analysis using CT images are not good enough to be used in clinical practice due to their inaccuracy. Some attempts at a solution transferred motion estimation concepts from other modalities to CT with moderate success. One such approach is described in F. Ammon et al., “CT-abgeleitete linksventrikuläre globale Belastung: ein direkter Vergleich mit der Speckle-Tracking-Echokardiographie,” [CT-derived left ventricular global strain: a direct comparison with Speckle Tracking Echocardiography (STE)], The International Journal of Cardio Imaging, vol. 35, no. 9, p. 1701-1707, 2019, doi: 10.1007/s10554-019-01596-8 and in S. J. Buss et al., “Quantitative Analysis of Left Ventricular Strain Using Cardiac Computed Tomography,” European Journal of Radiology, vol. 83, No. 3, e123-e130, 2014, doi: 10.1016/j.ejrad.2013.11.026.

Another approach, in which myocardial motion is estimated on the basis of a model adjusted to cardiac CT images taken at different times, appears the most promising in the prior art, even though the approach may not be fully automated, is limited to the left ventricle, and has specific inaccuracies. This approach is described in Z. Peled et al., “Automatisierte 4-dimensionale regionale Myokardbelastungsbewertung mittels kardialer Computertomographie,” [Automated 4-dimensional regional myocardial strain evaluation using cardiac computed tomography], The international Journal of Cardio Imaging, vol. 36, No. 1, p. 149-159, 2020, doi: 10.1007/s10554-019-01696-5 and in Y. Lamash et al., “Strain Analysis From 4-D Cardiac CT Image Data,” IEEE Transactions on Biomedical Engineering, vol. 62, No. 2, p. 511-521, 2015, doi: 10.1109/TBME.2014.2359244.

### SUMMARY

One or more example embodiments adapts 4D-CT imaging of the heart such that it is suitable for a cardiac deformation analysis.

### BRIEF DESCRIPTION OF THE DRAWINGS

One or more example embodiments is explained in more detail below with reference to the attached figures based on example embodiments. They show:

FIG. 1 a flow chart demonstrating a method for generating segmented, masked 4D image data of the heart according to an example embodiment of the invention,

FIG. 2 an image sequence illustrating step 1.I and step 1.II of the method demonstrated in FIG. 1 according to an example embodiment of the invention,

FIG. 3 a diagram also illustrating step 1.I and step 1.II of the method demonstrated in FIG. 1 according to an example embodiment of the invention,

FIG. 4 an image display illustrating step 1.III and step 1.IV of the method demonstrated in FIG. 1 according to an example embodiment of the invention,

FIG. 5 a schematic representation of an image data generating device according to an example embodiment of the invention,

FIG. 6 a flow chart demonstrating a cardiac deformation analysis method according to an example embodiment of the invention,

FIG. 7 a schematic representation of cardiac deformation analysis equipment according to an example embodiment of the invention, and

FIG. 8 a schematic representation of a computed tomography system according to an example embodiment of the invention.

### DETAILED DESCRIPTION

With the method according to one or more example embodiments for generating segmented, masked 4D image data of the heart, a 4D image data recording, preferably image data from an angiography, particularly preferably a coronary angiography, is provided of a patient's heart, preferably as CT imaging, which has 3D or 4D image data of the patient's heart. Coronary angiography is an angiography of the coronary vessels and thus is a special form of X-ray examination that images the coronary vessels. Contrast agents are usually used to make the blood vessels visible for imaging in angiography.

The method according to one or more example embodiments for generating segmented, masked 4D image data of the heart and the cardiac deformation analysis method according to one or more example embodiments described below is designed in particular as a computer-implemented method.

In addition to coronary angiography, all other methods for recording 4D data in which the muscle of a ventricle can be differentiated from the blood in the ventricle are also intended to be included. Consequently, such recording can also include CT recording with a high CT resolution without contrast agents. The recording can also include recording with transoesophageal 4D ultrasound. Alternatively, the 4D data recording can also include MRT imaging.

The first segmented 4D image data is generated based on the 4D image data, wherein the heart wall of a ventricle, preferably the left ventricle, is segmented. Use of the method according to one or more example embodiments on the left ventricle is preferred, as the condition of the left ventricle has the greatest impact on the functioning of the systemic circulation. The right ventricle has a much thinner wall than the left ventricle. The function of the pulmonary circulation influenced by the right ventricle is not quite as clinically relevant as the function of the systemic circulation. The imaging of the right ventricle must also explicitly be included at this point, however, albeit less prominently.

A method for ventricle segmentation is described in Y. Zheng et al., “Four-chamber heart modeling and automatic segmentation for 3-D cardiac CT volumes using marginal space learning and steerable features,” IEEE transactions on medical imaging, vol. 27, no. 11, pp. 1668-1681, 2008, doi: 10.1109/TMI.2008.2004421. The aforementioned document is incorporated into this patent application via reference thereto.

Time-resolved three-dimensional image data is used as 4D image data. The heart wall is formed by the so-called myocardium, the heart muscle tissue. The segmentation is designed so that the heart wall of the ventricle, preferably the left ventricle, is differentiated from the surroundings. As explained in more detail later, steps are preferably taken to highlight the heart wall of the ventricle, preferably the left ventricle, compared to the image areas surrounding the heart wall, so that a motion of the heart wall of the ventricle, especially the left ventricle, especially a contraction, dilation, or expansion of the heart wall, can be traced precisely and clearly.

The second segmented 4D image data is generated based on the first segmented 4D image data, wherein the epicardium and endocardium of the ventricle are segmented. The epicardium is the outermost layer or skin of the heart. It fuses with the myocardium. The endocardium is the innermost layer of the heart, which covers the entire inner surface of the heart like a smooth inner skin of the heart. The second segmented 4D image data preferably includes a highlighting of the endocardium and epicardium from the surroundings, designed in such a way that a motion of the endocardium and epicardium can be traced precisely and clearly.

Segmented, masked 4D image data is generated based on the second segmented 4D image data, wherein the interior of the ventricle, preferably the left ventricle, is covered. The interior of a ventricle, especially the left ventricle, includes the valve muscles of the heart valves, which also move when the ventricle in question is deformed. A motion of the valve muscles can wrongly be perceived as a motion of the heart wall of the ventricle in question, wherein the deformation analysis would be distorted.

The ventricle, preferably the left ventricle, and its contours are advantageously rendered much more visible and are clearly differentiated from the surroundings, wherein more precise surface mapping is facilitated amongst other things. The increase in the contrast of the heart structures also facilitates improved motion tracking.

By improving images of adjacent cardiac phases, the intensity-based registration between the images is better focused on the actual area of interest for the LV myocardial strain analysis or cardiac deformation analysis. The registration algorithms provide deformation fields from which intrinsic myocardial motion trajectories can be derived.

Intensity-based registration algorithms use smoothness restrictions to create realistic motion estimates. Motion around the surveyed subject can mislead the registration process and lead to inaccurate motion estimates for the subject. This effect can be eliminated by hiding misleading motions/objects in the image.

If the ventricle, preferably the left ventricle, is contracting, density increases within the heart muscle, which is indicated by increased brightness or contrast in this area. A contraction can be quantified with greater precision thanks to better utilization of the bandwidth of possible brightness values. Conversely, expansion or dilation can also be identified if a reduction in density is detected. In addition, the spacing between the epicardium and endocardium also changes during contraction and dilation, which is also easier to identify thanks to the segmentation described above.

Twisting, which occurs during a contraction, can also be identified where applicable.

The method according to one or more example embodiments for generating segmented, masked 4D image data of the heart is particularly advantageous when applied to low-contrast imaging methods that are to be used to perform a cardiac function analysis, especially a cardiac deformation analysis.

With the cardiac deformation analysis method according to one or more example embodiments, the method according to one or more example embodiments for generating segmented, masked 4D image data of the heart is performed first. Next, a deformation field is determined based on the segmented, masked 4D image data generated by the method. The dynamic 4D image data is registered with a reference image for this purpose.

The deformation field then has displacement vectors from pixels of a subject image to pixels of a reference image, which are caused by an elastic element of the registration method.

An intrinsic myocardial motion trajectory is determined on the basis of the determined deformation field. A myocardial deformation analysis of the ventricle, preferably the left ventricle, is performed on the basis of the determined intrinsic myocardial motion trajectory.

A more precise analysis of cardiac deformation is possible thanks to the improved image data on which the cardiac deformation analysis method is based, wherein the significance and accuracy of a prognosis of the health of a patient's heart can be improved.

The image data generating device according to one or more example embodiments has an input interface to receive 4D image data of the patient's heart. Part of the image data generating device according to one or more example embodiments is also a first segmentation unit for generating the first segmented 4D image data based on the 4D image data, wherein the heart wall of a ventricle, preferably the left ventricle, is segmented.

The image data generating device according to one or more example embodiments also has a second segmentation unit for generating the second segmented 4D image data based on the first segmented 4D image data, wherein the epicardium and endocardium of the ventricle, preferably the left ventricle, are segmented.

Furthermore, the image data generating device according to one or more example embodiments has a mask unit for generating segmented, masked 4D image data based on the second segmented 4D image data, wherein the interior of the ventricle, preferably the left ventricle, is covered. The image data generating device according to one or more example embodiments applies the advantages of the method according to one or more example embodiments to the generation of segmented, masked 4D image data of the heart.

The medical imaging system according to one or more example embodiments has a scanning unit for capturing measurement data, preferably projection data, for a patient and a control unit for controlling the scanning unit and for generating image data based on the measurement data, preferably projection data. Part of the medical imaging system according to one or more example embodiments is also an image data generating device according to one or more example embodiments. The imaging system according to one or more example embodiments applies the advantages of the method according to one or more example embodiments to the generation of segmented, masked 4D image data of the heart.

In particular, the features and advantages described in connection with the method according to one or more example embodiments can also be developed as corresponding subunits of the medical imaging system or the computer program product according to one or more example embodiments.

Conversely, the features and advantages described in connection with the medical imaging system according to one or more example embodiments and the computer program product according to one or more example embodiments can also be developed as corresponding method steps of the method according to one or more example embodiments.

The medical imaging system preferably includes a computed tomography system. The computed tomography system according to one or more example embodiments has a scanning unit for capturing projection data for a patient and a control unit for controlling the scanning unit and for generating image data based on the projection data. Part of the computed tomography system according to one or more example embodiments is also an image data generating device according to one or more example embodiments. The computed tomography system according to one or more example embodiments applies the advantages of the method according to one or more example embodiments to the generation of segmented, masked 4D image data of the heart.

Alternatively, the medical imaging system can also include an ultrasound imaging system with transoesophageal 4D ultrasound. Alternatively, the 4D data recording can also include MRT imaging.

The computer program product according to one or more example embodiments has program code sections that can be used to execute all the steps of the method according to one or more example embodiments for generating image data based on projection data or the cardiac deformation analysis method, if the program is executed in a control device of a medical imaging system, preferably a computed tomography system.

Predominantly software-based realization has the advantage that medical imaging systems, preferably computed tomography systems, or their control devices, can already be easily retrofitted to work in the manner according to one or more example embodiments via a software update.

The majority of the aforementioned components of the image data generating device according to one or more example embodiments can be realized, wholly or partially, in the form of software modules in a processor of a corresponding computer system, e.g., of a control device of a medical imaging system or a computer that is used to control such a system. Predominantly software-based realization has the advantage that even previously used computer systems can already be easily retrofitted to work in the manner according to one or more example embodiments via a software update. In this respect, the object is also achieved through a corresponding computer program product with a computer program that can be loaded directly in a computer system, with program sections, in order to execute the steps of the method according to one or more example embodiments for generating segmented, masked image data of the heart based on the projection data or the steps of the cardiac deformation analysis method, if the program is executed in the computer system. In addition to the computer program, such a computer program product can include additional elements, e.g., documentation and/or additional components, including hardware components, e.g., hardware tools (dongles etc.), for use of the software.

A computer-readable medium can be used, e.g., a memory stick, a hard drive, or other transportable or integral data carrier, on which the program sections of the computer program are stored that can be read and executed by a computer system, for transport to the computer system or to the control device and/or for storage in or on the computer system or control device. The computer system can have, e.g., one or more cooperating microprocessors or the like.

The dependent claims and the following description include particularly advantageous embodiments and developments of one or more example embodiments in each case. In particular, the claims of one claim category can also be developed analogous to the dependent claims of another claim category. Within the scope of the invention the various features of the different example embodiments and claims can also be combined into new example embodiments.

In one variant of the method according to one or more example embodiments for generating segmented, masked 4D image data of the heart, the step of generating the first segmented 4D image data includes the production of a mask of the wall of the left ventricle. The mask of the wall of the left ventricle marks the area that is relevant for a cardiac deformation analysis. If visibility of this area is improved, it is easier to register the heart walls and their surfaces, especially the epicardium and endocardium. Thus, the accuracy and reliability of a deformation analysis of the left ventricle can be improved on the basis of a registration of the heart walls and surfaces of the 4D image data improved in this way.

The mask of the wall of the left ventricle is preferably produced by applying a four-chamber segmentation algorithm to the 4D image data. The left ventricle can advantageously be segmented from the other chambers of the heart using a four-chamber segmentation algorithm.

A contrast-rich mask image is produced in one variant of the method according to one or more example embodiments for generating segmented, masked 4D image data of the heart. In the process, the voxels within the mask are determined, the gray values or brightness values of which deviate from a statistical mean value by no more than a predetermined value. The gray values are mapped onto contrast-rich gray values, which fall between the gray value 0 and the sum of the statistical mean value and the predetermined value. Advantageously, the scale of the brightness values is better utilized in the area of the mask, in order to make textures and contrasts clearer.

In a preferred variant of the method according to one or more example embodiments for generating segmented, masked 4D image data of the heart, the predetermined value is two standard deviations from the mean value. In a normal distribution, 95 percent of the values of the distribution fall within the value range that is limited by two standard deviations around the mean value. The value range of the possible brightness values is advantageously defined so that most of the values of the voxels of the mask area within the mask fall within this range.

In a particularly preferable embodiment of the method according to one or more example embodiments for generating segmented, masked 4D image data of the heart, the first segmented 4D image data is produced by combining the contrast-rich mask image with the 4D image data using weighting. The image data for the surroundings of the mask area is advantageously retained, and the contrast in the mask area is improved by adding the 4D image data with the higher contrast within the mask area.

In a preferred variant of the method according to one or more example embodiments for generating segmented, masked 4D image data of the heart, the weighted combination is effected at a predetermined percentage. The original image information is advantageously also included in the segmented image data. In particular, the voxels outside the wall of the left ventricle are unchanged.

In a preferable embodiment of the method according to the invention for generating segmented, masked 4D image data of the heart, the percentage between 20 and 40 percent for the contrast-rich mask image is preferably around 30 percent relative to the segmented 4D image data. This means that the contrast-rich mask image is taken at 20 to 40 percent and the original image at 60 to 80 percent for the segmented, masked 4D image data. With the value of 30 percent, the contrast-rich mask image is weighted at 30 percent and the original 4D image data at 70 percent. A dominant portion of the original image information is advantageously included in the segmented, masked 4D image data of the heart.

The second segmented 4D image data is preferably generated by highlighting the contrast of the epicardium and endocardium of the left ventricle. Advantageously, the surfaces of the left ventricle are clearly highlighted, which makes the registration process easier and facilitates precise motion tracking for the surfaces of the left ventricle.

When generating the second segmented 4D image data based on the first segmented 4D image data, the brightness values of the area segmented as the epicardium and endocardium are preferably changed to a mean value plus twice the standard deviation from this mean value. Advantageously, the surfaces of the left ventricle are rendered extremely visible.

An interface gain factor is preferably defined, in order to maintain the texture information on the surface of the left ventricle. This interface gain factor is used to define the percentage at which the voxel values within the surface are set to the aforementioned mean value plus twice the standard deviation from this mean value and the percentage at which the original values for voxels in the surface are retained in the resulting image. The gray values in the surface of the left ventricle can be multiplied by an additional factor that is greater than 1. This renders the endocardium and epicardium extremely visible in the resulting image and makes intensity-based registration easier.

It is also preferable for the step of generating the segmented, masked 4D image data to include the production and inversion of a mask for the intraventricular blood lumen, which includes the endocardial tissue. The mask for the intraventricular blood lumen is inverted so that the entire blood-filled volume or blood lumen is covered. The section of the left ventricle to be imaged is advantageously segmented or separated from the intraventricular blood lumen. As already stated, the interior of a ventricle includes the valve muscles of the heart valves, which also move when there is deformation of the ventricle. A motion of the valve muscles can wrongly be perceived as a motion of the heart wall of the ventricle, wherein the deformation analysis would be distorted. Thus, coverage of the interior of the ventricle is advantageous for a cardiac deformation analysis.

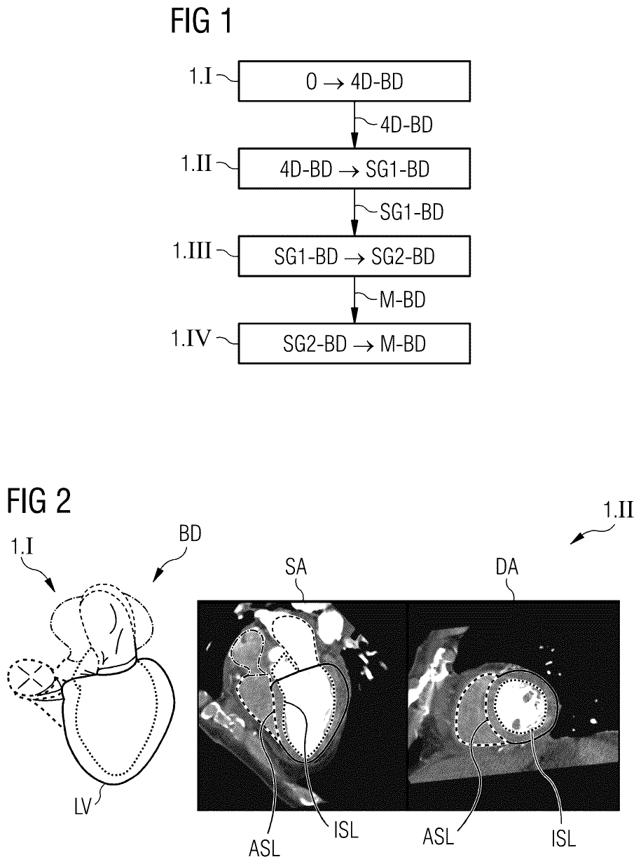

FIG. 1 shows a flow chart that demonstrates a method for generating segmented, masked 4D image data M-BD of the heart according to an example embodiment of the invention.

In step 1.I, a coronary angiography is provided of the heart of the patient O, which has 4D image data 4D-BD of the heart of the patient O.

The first segmented 4D image data SG1-BD is generated in step 1.II based on the 4D image data 4D-BD, wherein the heart wall of the left ventricle of the heart of the patient O is segmented.

The second segmented 4D image data SG2-BD is generated in step 1.III based on the first segmented 4D image data SG1-BD, wherein the epicardium and endocardium of the left ventricle are segmented.

Finally, segmented, masked 4D image data M-BD is generated in step 1.IV based on the second segmented 4D image data SG2-BD, wherein the interior of the left ventricle is covered.

FIG. 2 shows an image sequence that illustrates step 1.I and step 1.II of the method demonstrated in FIG. 1 according to an example embodiment of the invention. The image sequence in FIG. 2 includes three partial images, of which the partial image shown on the left illustrates image data BD of a perspective view of the heart with a left ventricle LV. A center partial image shows a longitudinal axis view SA of the left heart ventricle, and a right partial image shows a short axis view DA of the left heart ventricle. Segmentation lines are shown in the center partial image and the right partial image, which demarcate the interior and exterior of the heart chamber muscle. The outer dashed segmentation lines ASL demarcate the muscle tissue outwards to the pericardium, while the inner dotted segmentation lines ISL demarcate the muscle tissue inwards to the endocardium.

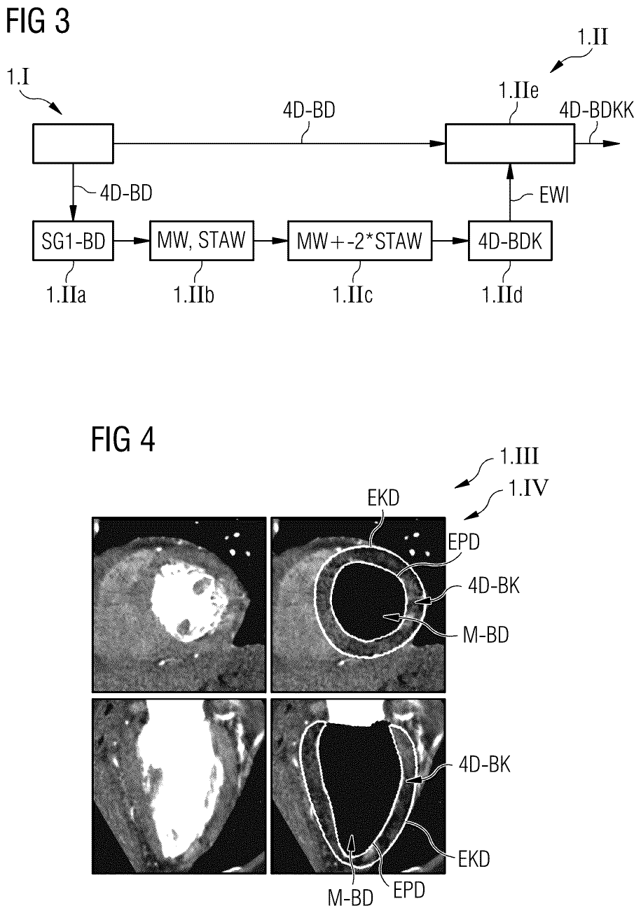

FIG. 3 shows a diagram that illustrates step 1.I and step 1.II of the method demonstrated in FIG. 1 according to an example embodiment of the invention. The already described imaging of a patient's heart is effected in step 1.I, wherein 4D image data 4D-BD of the heart of a patient O is obtained.

In addition to the segmentation of the left ventricle, which is illustrated in FIG. 2, step 1.II also includes a modified image display, wherein the contrast of the display of the segmented area, i.e., the left ventricle, is increased.

A mask is produced in substep 1.IIa, which covers the wall of the left ventricle. The brightness values or gray values of the voxels covered by the mask are then statistically analyzed in substep 1.IIb. In the process, a mean value for the brightness values or gray values is determined, measured in Hounsfield units (HU). A standard deviation from the mean value is also determined in step 1.IIb. In a normal distribution, the brightness values of 95 percent of all voxels fall within a value range that is characterized by the mean value MW plus/minus twice the standard deviation STAW from the mean value MW as boundary values.

In substep 1.IIc, the brightness values of the voxels within the mask are limited to this value range. In particular, brightness values outside the stated value range are set to the boundary values, i.e., the aforementioned mean value MW plus/minus twice the standard deviation STAW.

In substep 1.IId, the brightness values of the voxels in the mask area are redistributed to an expanded value interval EWI, which ranges from the value 0 to a value that is formed from the mean value MW and twice the standard deviation STAW. 4D image data 4D-BDK with increased contrast in the mask area is formed in this way.

In substep 1.Ile, the original 4D image data 4D-BD and the 4D image data 4D-BDK with increased contrast generated in substep 1.IId are combined or overlapped weighted with a weighting factor, wherein combined 4D image data 4D-BDKK with increased contrast is produced. The weighting factor indicates the percentages at which the 4D image data 4D-BDK with increased contrast in the mask area and the original 4D image data 4D-BD in the mask area should be combined. The voxels of the original 4D image data 4D-BD outside the mask area are retained unchanged.

FIG. 4 shows an image display that illustrates step 1.III and step 1.IV of the method demonstrated in FIG. 1 according to an example embodiment of the invention. In step 1.III, the peripheral areas of the ventricle are provided with a kind of surface mask. The mask can be determined by a morphological dilation operation. The peripheral areas are determined in the process, starting from a point inside the mask or the 4D image data 4D-BDK with increased contrast in the mask area that was generated in step 1.II. The brightness values of the surface mask are then increased to the maximum value of the previously produced value range, i.e., to the mean value plus twice the standard deviation STAW. In order to maintain the texture information in the surface mask, the original brightness values for the pixels in the area of the surface mask can also be combined with the increased brightness values with weighting. The endocardium EKD and epicardium EPD are illuminated in this way. In step 1.IV, segmented, masked 4D image data M-BD is generated by shadowing the interior of the left ventricle. I.e., the interior of the left ventricle, which appears light due to the contrast agent and the resulting increased X-ray absorption, is inverted with regard to its X-ray weakness and thus appears dark.

FIG. 5 shows a schematic representation of an image data generating device 50 according to an example embodiment of the invention. The image data generating device 50 has an input interface 51, which is set up to receive 4D image data 4D-BD of the patient's heart.

Part of the image data generating device 50 is also a first segmentation unit 52, which is set up to generate the first segmented 4D image data SG1-BD based on the 4D image data 4D-BD, wherein the heart wall of the left ventricle is segmented.

The image data generating device 50 also includes a second segmentation unit 53, which is set up to generate the second segmented 4D image data SG2-BD based on the first segmented 4D image data SG1-BD, wherein the epicardium and endocardium of the left ventricle are segmented.

Moreover, the image data generating device 50 has a masking unit 54, which is set up to generate segmented, masked 4D image data M-BD based on the second segmented 4D image data SG2-BD, wherein the interior of the left ventricle is covered.

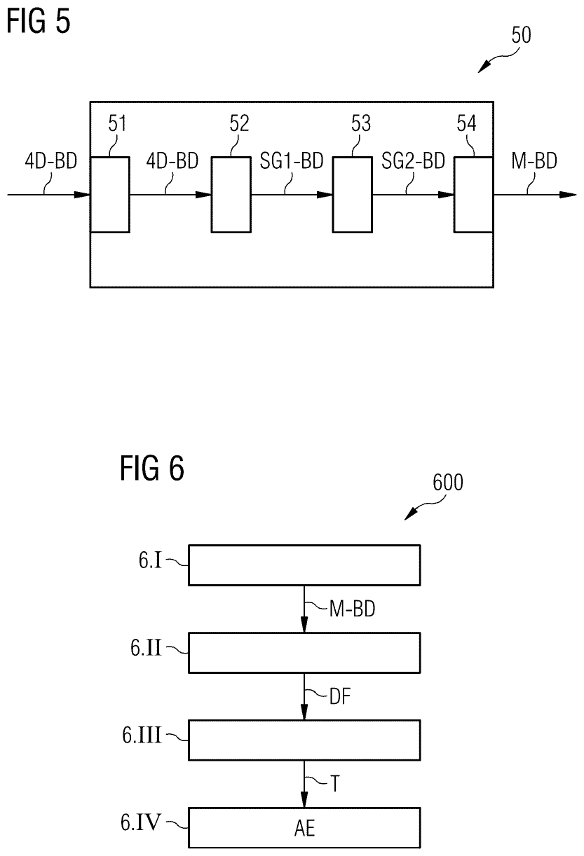

FIG. 6 shows a flow chart 600 that demonstrates a cardiac deformation analysis method according to an example embodiment of the invention.

The method demonstrated in FIGS. 1 to 4 for generating segmented, masked 4D image data M-BD of the heart according to an example embodiment of the invention is performed in step 6.I.

In step 6. II, a deformation field is determined based on the segmented, masked 4D image data M-BD generated by the method.

In step 6. III, an intrinsic myocardial motion trajectory T is determined based on the determined deformation field.

In step 6. IV, a myocardial deformation analysis of the left ventricle is performed based on the determined intrinsic myocardial motion trajectory, and a result AE of the deformation analysis is produced.

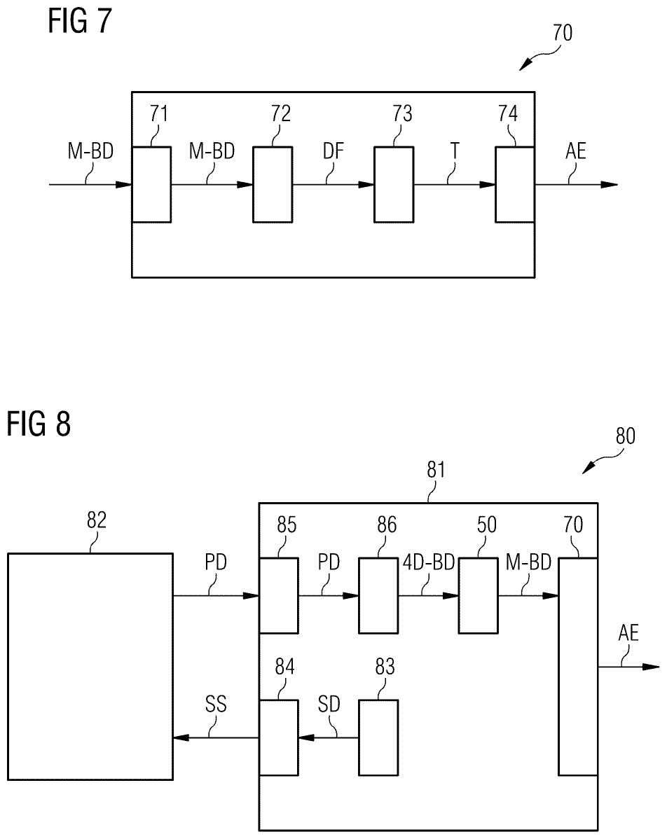

FIG. 7 shows a schematic representation of cardiac deformation analysis equipment 70 according to an example embodiment of the invention.

The cardiac deformation analysis equipment 70 has an input interface 71 to receive segmented, masked 4D image data M-BD of the heart. The segmented, masked 4D image data M-BD of the heart is received by an image data generating device 50 as shown in FIG. 5. Part of the cardiac deformation analysis equipment 70 is also a field determining unit 72, which is set up to determine a deformation field DF based on the segmented, masked 4D image data M-BD generated by the method.

Part of the cardiac deformation analysis equipment 70 is also a trajectory determining unit 73, which is set up to determine an intrinsic myocardial motion trajectory T based on the determined deformation field DF.

Moreover, part of the cardiac deformation analysis equipment 70 is an analysis unit 74, which is set up to perform a deformation analysis of the left ventricle based on the determined intrinsic myocardial motion trajectory T and produce a result AE of the deformation analysis.

FIG. 8 shows a schematic representation of a computed tomography system 80 according to an example embodiment of the invention.

The computed tomography system 80 has a control device 81 and a scanning unit 82 controlled by the control device 81. The control device 81 has a control unit 83 for generating control data SD and a control interface 84 for transmitting control signals SS, which are generated based on the control data SD, to the scanning unit 82. Part of the control device 81 is also an input interface 85, which is set up to receive projection data PD from the scanning unit 82. The projection data PD is transmitted to a reconstruction unit 86, which is also part of the control device 81 and is set up to reconstruct 4D image 4D-BD data of the heart of a patient (not shown) lying in the scanning unit 82. Part of the control device 81 is also the image data generating device 50 already shown in FIG. 5, which is set up to generate mask image data M-BD of the heart based on the 4D image data 4D-BD. The control device 81 also includes the cardiac deformation analysis equipment 70 shown in FIG. 7, which is set up to perform a deformation analysis based on the generated segmented, masked 4D image data M-BD and to produce result data AE of the analysis.

Finally, it is noted once again that the invention and developments described in detail above merely relate to example embodiments, and that the basic principle can also be modified by an expert in broad areas without leaving the field of the invention, insofar as it is specified by the claims. It must be noted in this context that the features of all example embodiments or developments disclosed in figures can be used in any combination.

Moreover, for the sake of completeness it is noted that use of the indefinite article “a” or “an” does not prevent the features concerned being present multiple times. Likewise, the term “unit” does not exclude the possibility that this comprises multiple components, which may be also be spatially distributed. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that encompasses relationship a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, terms all (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for generating segmented, masked 4D image data of a heart, the method comprising:
providing a 4D image of a heart of a patient, the 4D image having 4D image data of the heart of the patient;
generating first segmented 4D image data based on the 4D image data, wherein a heart wall of a ventricle is segmented;
generating second segmented 4D image data based on the first segmented 4D image data, wherein an epicardium and an endocardium of the ventricle are segmented; and
generating the segmented, masked 4D image data based on the second segmented 4D image data, wherein an interior of the ventricle is covered.

2. The method of claim 1, wherein the ventricle comprises a left ventricle.

3. The method of claim 1, wherein the generating the first segmented 4D image data includes producing a mask of the heart wall of the ventricle.

4. The method of claim 3, wherein the producing produces the mask by applying a four-chamber segmentation algorithm to the 4D image data.

5. The method of claim 3, wherein the generating the segmented, masked 4D image data includes,
generating a contrast-rich mask image, wherein voxels within a mask are determined, gray values within the mask deviate from a mean value by no more than a predetermined value, and the gray values are mapped onto contrast-rich gray values, the contrast-rich gray values are between the gray value 0 and a sum of the mean value and the predetermined value.

6. The method of claim 5, wherein the predetermined value is two standard deviations from the mean value.

7. The method of claim 5, wherein the generating the first segmented 4D image data generates the first segmented 4D image data by combining the contrast-rich mask image with the 4D image data using weighting.

8. The method of claim 7, wherein the weighted combination is effected at a predetermined percentage.

9. The method of claim 8, wherein the percentage between 20 and 40 percent for the contrast-rich mask image is around 30 percent relative to the first segmented 4D image data.

10. The method of claim 1, wherein at least one of
the generating the second segmented 4D image data generates the second segmented 4D image data by highlighting a contrast of the epicardium and endocardium of the ventricle, or
the generating the segmented, masked 4D image data includes producing and inverting an intraventricular mask.

11. A cardiac deformation analysis method, the method comprising:
performing the method of claim 1;
determining a deformation field based on the segmented, masked 4D image data;
determining an intrinsic myocardial motion trajectory based on the determined deformation field; and
performing a myocardial deformation analysis of the left ventricle based on the determined intrinsic myocardial motion trajectory.

12. An image data generating device comprising:
an input interface configured to receive 4D image data of a heart of a patient;
a first segmentation unit configured to generate first segmented 4D image data based on the 4D image data, wherein a heart wall of a ventricle of the heart is segmented;
a second segmentation unit configured to generate second segmented 4D image data based on the first segmented 4D image data, wherein an epicardium and an endocardium of the ventricle are segmented; and
a mask unit configured to generate segmented, masked 4D image data based on the second segmented 4D image data, wherein an interior of the ventricle is covered.

13. A medical imaging system, comprising:
a scanning unit to capture measurement data of the patient;
a control device to control a scanning unit and to generate the 4D image data based on the measurement data; and
the image data generating device of claim 12.

14. A non-transitory computer program product with a computer program, when executed by a medical imaging system, cause the medical imaging system to perform the method of claim 1.

15. A non-transitory computer-readable medium on which are stored program sections, executed by a medical imaging system, cause the medical imaging system to perform the method of claim 1.

16. The method of claim 4, wherein the generating the segmented, masked 4D image data includes,
generating a contrast-rich mask image, wherein voxels within a mask are determined, gray values within the mask deviate from a mean value by no more than a predetermined value, and the gray values are mapped onto contrast-rich gray values, the contrast-rich gray values are between the gray value 0 and a sum of the mean value and the predetermined value.

17. The method of claim 6, wherein the generating the first segmented 4D image data generates the first segmented 4D image data by combining the contrast-rich mask image with the 4D image data using weighting.

18. The method of claim 5, wherein at least one of
the generating the second segmented 4D image data generates the second segmented 4D image data by highlighting a contrast of the epicardium and endocardium of the ventricle, or
the generating the segmented, masked 4D image data includes producing and inverting an intraventricular mask.
